(19) United States
(12) Patent Application Publication
(10) Pub. No.: US 2026/0256353 A1
(43) Pub. Date: Sep. 3, 2026

(54) OPTICAL SYSTEM TO OBJECTIVELY IDENTIFY AND TRACK ACCOMMODATION OF AN INTRAOCULAR LENS AND METHODS OF USE

(71) Applicant: ForSight Vision6, Inc., South San Francisco, CA (US)

(72) Inventors: Austin J. Roorda, South San Francisco, CA (US); Matthew Clarke, South San Francisco, CA (US); Ayman Naseri, South San Francisco, CA (US)

(21) Appl. No.: 18/730,713

(22) PCT Filed: Jan. 20, 2023
(86) PCT No.: PCT/US2023/060967

§ 371 Date: Jul. 19, 2024

Related U.S. Application Data
(60) Provisional application No. 63/301,846, filed on Jan. 21, 2022.

(51) Int. Cl. A61B 3/09; A61B 3/00; A61B 3/103; A61B 3/11; A61B 3/14

(52) U.S. Cl. CPC A61B 3/09; A61B 3/0025; A61B 3/103; A61B 3/112; A61B 3/145

(57) ABSTRACT

An optical system to objectively measure accommodation in a patient including an infrared light source configured to provide retro-illumination light to a retina of an eye during a test; a wavefront camera configured to continuously record Shack-2023/141552 Hartmann centroids from the eye during the test providing a wavefront camera output; and an infrared-sensitive pupil camera configured to continuously record video of the eye from the retro-illumination light returned from the eye during the test providing a pupil camera output. The wavefront camera and the pupil camera are operatively coupled to synchronously or near synchronously operate at a sampling rate during the test. Related devices and methods are provided.

### CROSS-REFERENCE TO RELATED APPLICATIONS

This application claims the benefit of priority under 35 U.S.C. § 119(e) to U.S. Provisional Patent Application Ser. No. 63/301,846, filed Jan. 21, 2022. The disclosure of the application is incorporated by reference in its entirety.

### BACKGROUND

Described herein are methods, devices, and systems that pertain generally to ophthalmic diagnostics, particularly for the identification of optical vision deficiencies and measuring accommodation of an intraocular lens.

A healthy, young human eye can focus an object in far or near distance, as required. The capability of the eye to change back and forth from near vision to far vision is called accommodation. Accommodation occurs when the ciliary muscle contracts to thereby release the resting zonular tension on the equatorial region of the capsular bag. The release of zonular tension allows the inherent elasticity of the lens to alter to a more globular or spherical shape, with increased surface curvatures of both the anterior and posterior lenticular surfaces.

The human eye 10 includes a cornea 12, iris 14, ciliary muscle 18, zonules 20, a lens 21 contained within a capsular bag 22 (FIGS. 1A-1B). Accommodation occurs when the ciliary muscle 18 contracts to thereby release the resting zonular tension on the equatorial region of the capsular bag 22. The release of zonular tension allows the inherent elasticity of the lens 21 to alter to a more globular or spherical shape, with increased surface curvatures of both the anterior lenticular surface and posterior lenticular surface. In addition, the human lens can be afflicted with one or more disorders that degrade its functioning in the vision system. A common lens disorder is a cataract which consists of the opacification of the normally clear, natural crystalline lens matrix. The opacification can result from the aging process but can also be caused by heredity, diabetes, or trauma. In a cataract procedure, the patient's opaque crystalline lens is replaced with a clear lens implant or intraocular lens (IOL) 30 (FIG. 1B). In conventional extracapsular cataract surgery as depicted in FIG. 1B, the crystalline lens matrix is removed leaving intact the thin walls of the anterior and posterior capsules 22 together with zonular ligament connections 20 to the ciliary body and ciliary muscles 18. The crystalline lens core is removed by phacoemulsification through a curvilinear capsulorhexis as illustrated in FIG. 1B, i.e., the removal of an anterior portion of the capsular sac. FIG. 1B depicts a conventional IOL 30 just after implantation in the capsular bag 22.

A light beam from a light source can enter the eye and refract into the cornea 12, pass through the anterior chamber 25, the iris 14 through the pupil, and reach the lens 21. After refracting into the lens 21, light passes through the vitreous chamber 26, and strikes the retina 28, which detects the light and converts it to an electric signal transmitted through the optic nerve to the brain. The vitreous chamber 26 contains vitreous humor, a clear liquid disposed between the lens 21 and the retina 28. The cornea 12 has a corneal thickness CT between the anterior and posterior surfaces of the cornea. The anterior chamber 25 has an anterior chamber depth ACD between the anterior surface of the lens 21 and the anterior surface of the cornea 12. The lens has a lens thickness LT between the anterior and posterior surfaces of the lens. The eye also has an axial length AXL between the anterior surface of the cornea and the retina 28.

Accommodating IOLs are beneficial for patients not suffering from cataracts, but who wish to reduce their dependency on glasses and contacts to correct their myopia, hyperopia and presbyopia. Intraocular lenses used to correct large errors in myopic, hyperopic, and astigmatic eye are called “phakic intraocular lenses” and are implanted without removing the crystalline lens. In some cases, aphakic IOLs (not phakic IOLs) are implanted via lens extraction and replacement surgery even if no cataract exists. During this surgery, the crystalline lens is extracted and an IOL replaces it in a process that is very similar to cataract surgery. Refractive lens exchange, like cataract surgery, involves lens replacement, requires making a small incision in the eye for lens insertion, use of local anesthesia and lasts approximately 30 minutes.

Premium IOLs such as accommodating IOLs, multifocals, torics, and the like can be associated with visual phenomena such as glare, halos, and night vision problems that can be worsened by any refractive error. Thus, refractive predictability is important for improved visual outcomes offered by these lenses. Traditional vision diagnostic tools are known to improve the refractive results including auto-refractors, wavefront aberrometry, keratometry, and topography measurement. Corneal power in abnormal corneas can impact the accuracy of these tools and the postoperative visual results.

A wavefront aberrometer is a device for objectively measuring refractive power by detecting higher-order and lower-order aberrations of an optical wavefront. Meaning, the refractive errors can be identified automatically by the way the light waves travel through the eye. Wavefront aberrometers have been used for eye aberration measurement by directing a narrow beam of light to the retina of an eye and sensing the optical wavefront coming out from the eye. For a relaxed emmetropic eye or a relaxed eye with aberrations completely corrected, the optical wavefront coming out from the eye is planar. If, on the other hand, the eye has optical aberrations, the wavefront coming out from the eye in a relaxed state will be non-planar. Although wavefront measurement systems have been used to measure the refractive errors and higher order aberrations of the eye, commercially available ophthalmic wavefront measurement devices generally only produce a static snapshot display of the wavefront map of the measurement. Some wavefront measurement systems incorporate pupillometry to assess the physiological alignment of the optical structures in the eye. However, pupil diameter changes during accommodation of the eye as does the pupillary center.

### SUMMARY

Provided are improved optical systems that actively track and continuously measure patient refraction and improve the accuracy of optical diagnostic systems to assess dynamically changing aberration patterns. The systems are compatible with a target presentation, namely unobstructed binocular view of a high resolution target, and maximally elicits accommodation that can be presented at varying viewing distances.

In an aspect, described is an optical system to objectively measure accommodation in a patient. The system includes an infrared light source configured to provide retro-illumination light to a retina of an eye during a test; a wavefront camera configured to continuously record Shack-Hartmann centroids from the eye during the test providing a wavefront camera output; and an infrared-sensitive pupil camera configured to continuously record video of the eye from the retro-illumination light returned from the eye during the test providing a pupil camera output. The wavefront camera and the pupil camera are operatively coupled to synchronously or near synchronously operate at a sampling rate during the test.

The wavefront camera and the pupil camera can operate synchronously within a tolerance of less than 100 milliseconds. The system can further include a computer system operatively coupling the pupil camera and the wavefront camera. The computer system can be programmed to index the wavefront camera output to an analytical zone of the pupil camera output independent of a pupil diameter of the eye. The computer system can be programmed to index the wavefront camera output to a structure of the eye visible on the pupil camera output. The structure of the eye does not change size during the test. The structure of the eye includes a man-made structure implanted in the eye including at least a portion of an intraocular lens or a natural structure of the eye including a limbus. The sampling rate can be at a frequency greater than about 10 Hz. The infrared light source can be an infrared laser or a super-luminescent diode. The infrared light source can be configured to provide a collimated light or probe beam during the test. The infrared light source can include a center wavelength of about 875 nm and a spectral bandwidth of about 855 nm-895 nm.

The system can further include a physical target positionable a distance away from the eye and visible by the patient under binocular, unimpeded viewing. The system can further include a dichroic filter positioned between the patient and the physical target. The dichroic filter can be a hot mirror.

In an interrelated implementation, provided is a method of using an optical system to assess patient refraction including directing infrared light from an infrared light source onto a retina of an eye during a test; continuously recording Shack-Hartmann centroids from the eye with a wavefront camera at a sampling rate during the test providing a wavefront camera output; and continuously recording video of the eye from the retro-illumination light returned from the eye at the sampling rate during the test by an infrared-sensitive pupil camera providing a pupil camera output.

The method can further include obtaining a calibration still shot of the eye from the pupil camera and obtaining a calibration still shot of the eye from the wavefront camera. The method can further include selecting an optical zone of interest from the pupil camera output using data processing software; and interpreting wavefront results to assess accommodative dynamics based on the optical zone of interest. The patient can be pseudophakic and have an intraocular lens. The intraocular lens can be monofocal, multifocal, EDoF, Toric, or accommodating. The patient can be phakic.

In an interrelated implementation, provided is a method of assessing patient refraction. The method includes performing gross alignment of an optical system including a physical target positionable during a test between a first distance and a second distance; an illuminating light source; a measurement light source configured to project a laser spot; an infrared-sensitive pupil camera; and a wavefront camera. The method includes projecting light from the illuminating light source of the optical system towards a patient so an eye to be measured is in view of the pupil camera and the wavefront camera. The method includes aligning the distance target with the laser spot visible to the patient; aligning the near target with the laser spot visible to the patient; and performing the test on the eye while operating the wavefront camera and the pupil camera at a sampling rate to obtain pupil camera output and wavefront camera output that is near synchronous.

Performing the test can include positioning the physical target for a period of time at the first distance and positioning the physical target for a period of time at the second distance while obtaining the pupil camera output and the wavefront camera output. The method can further include spatially calibrating the pupil camera output to the wavefront camera output to narrow an analytical range of the system to capture a full accommodative amplitude of the eye. Performing the test can include directing a probe beam from the measurement light source and capturing a wavefront by the wavefront camera while the pupil camera records the eye. The method can further include performing a test on a fellow eye. The method can further include selecting calibration markers on a calibration still frame captured from the wavefront camera output and selecting calibration markers on a calibration still frame captured from the pupil camera output. The method can further include selecting a zone of interest on a representative frame captured from the pupil camera output to identify an analysis zone. The method can further include isolating the analysis zone using data processing software. The method can further include performing extended analysis of wavefront data from the analysis zone selected using the data processing software. The method can further include interpreting wavefront results to assess accommodative dynamics of the eye. The method can further include performing gross alignment comprises aligning the physical target with a visual axis of the eye being measured. The physical target can be aligned with the eye being measured and the fellow eye converges.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

Generally speaking the figures are exemplary and are not to scale in absolute terms or comparatively but are intended to be illustrative. Relative placement of features and elements is modified for the purpose of illustrative clarity.

FIG. 1A is a cross-sectional view of a human eye in schematic;

FIG. 1B is a cross-sectional view of a human eye having an intraocular lens implanted;

FIG. 2 schematically illustrates a system for measurement of refraction of a lens in a patient;

FIG. 3 illustrates a structural configuration of the system of FIG. 2;

FIG. 4A is a top down view of an accommodating intraocular lens in schematic;

FIG. 4B is a cross-sectional view of an accommodating intraocular lens taken along line B-B of FIG. 5A;

FIGS. 5A-5B are still frames captured by the wavefront camera and pupil camera, respectively, prior to a measurement and using the system of FIG. 2 overlaid with spatial calibration markers;

FIG. 6A shows a still frame captured by the wavefront camera following a measurement using the system of FIG. 2;

FIG. 6B shows a retro-illumination still frame captured by the pupil camerafollowing a measurement using the system of FIG. 2;

FIG. 6C illustrates the analysis of full pupil showing attenuated signal of accommodation of Strehl Defocus (o) by RMS Defocus (x);

FIG. 7A shows a still frame captured by the wavefront camera following a measurement using the system of FIG. 2 where the central, dynamic optic of an IOL is selected for analysis;

FIG. 7B shows a retro-illumination still frame captured by the pupil camera following a measurement using the system of FIG. 2 where the central, dynamic optic of an IOL is selected for analysis;

FIG. 7C illustrates the analysis of the central, dynamic optic of the IOL resulted in full signal of accommodation of Strehl Defocus (o) by RMS Defocus (x);

FIG. 8 schematically illustrates components of a simplified computer system for use in the measurement components of the system of FIG. 2;

FIG. 9 is a block diagram illustration of a method of using the system to perform a test according to an implementation;

FIG. 10 is a block diagram illustration of a method of analyzing the raw data captured by the system of FIG. 9.

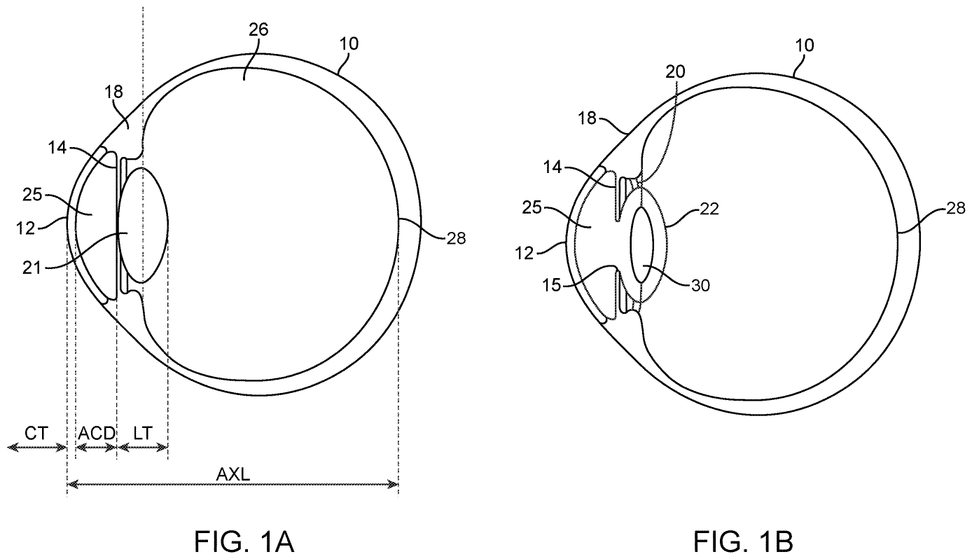

### DETAILED DESCRIPTION

Conventional wavefront aberrometers for human eye wavefront characterization are generally designed to take a snapshot or several snapshots of a patient's eye wavefront without any real-time feedback and adjustment of the readings. Some diagnostic systems may incorporate pupil tracking for indexing. During accommodation, the lens becomes thicker and more convex, the pupil constricts, and the eyes converge. The thickness of the lens refracts the light to form an image on the retina. Constriction of the pupil sharpens that focus by only allowing a few rays to pass through. Binocular convergence of the eyes is an important driver of accommodation. The shape change of pupils during accommodation and convergence of the eyes make it difficult to measure accommodation because the pupil serves as an ever-changing index.

The Grand Seiko WAM-5550 (RyuSyo Industrial Co., Lt., Kagawa, Japan) is a binocular accommodation auto-refractometer/keratometer for continuous measurement of accommodation that uses autorefraction based on the projection of a real target. The system incorporates continuous measurement with binocular direct viewing of the target and monocular measurement. The projection is a fixed diameter segmented circle. There is limited information in the autorefraction data (sphere, cylinder, and axis), no active tracking, and the about 2 mm diameter segmented circle is not ideal for certain IOLs. The Power Ref3 by PlusOptix uses photorefraction with infrared light. Photorefraction measures all of the light passing through the pupil and thus, cannot isolate the measurement to certain portions of an IOL. Additionally, the measurement generally requires >3.0 mm pupil, which can be a limitation for use in elderly post-cataract patients. The Power Ref3 system includes continuous measurement with binocular viewing of a real target, binocular measurement, and pupil/eye tracking. The accuracy and precision of the photorefraction data are not as strong as that of a Shack-Hartmann Wavefront system, particularly in pseudophakic patients with small pupils. Photorefraction systems generally require subject-specific calibrations. The Eye Refract by Visionix is a fully automated autorefractor/keratometer incorporating a Shack-Hartmann Wavefront aberrometer. Two aberrometers and two Shack-Hartmann sensors run simultaneously and perform static binocular refraction measurement in real time with binocular viewing of real targets. The Eye Refract system includes adjustable measurement diameter (artificial pupil), but has no ability to isolate and track a portion of an IOL. The HD Analyzer by Visiometrics is designed to provide clinicians with an objective measure of an eye's optical quality. The system measures the Point Spread Function of an eye with monocular view of a simulated target to achieve static measurements. The iTrace by Tracey Technologies incorporates a Ray Trace based Wavefront system. The system incorporates monocular view through a window that allows a patient to view actual distance and near targets. The iTrace data are static measurements and can be noisy in pseudophakic patients. The iTrace system allows for the static measurement of a particular region of the eye or IOL.

The devices, systems, and methods described herein allow for objectively measuring and tracking refraction errors of an eye. The devices, systems, and methods diagnose the refractive properties of the eyes of a patient and identify and characterize high-order aberrations. The devices, systems, and methods effectively gauge and characterize the actual effect of an overall prescription. The systems can be used on healthy (phakic) eyes as well as pseudophakic eyes implanted with any of a variety of IOL including monofocal, multifocal, EDoF, Toric, accommodating, or other lens. Although the system described herein is described in the context of patients having an accommodating IOL, other types of lenses including a natural lens of a patient is considered as well.

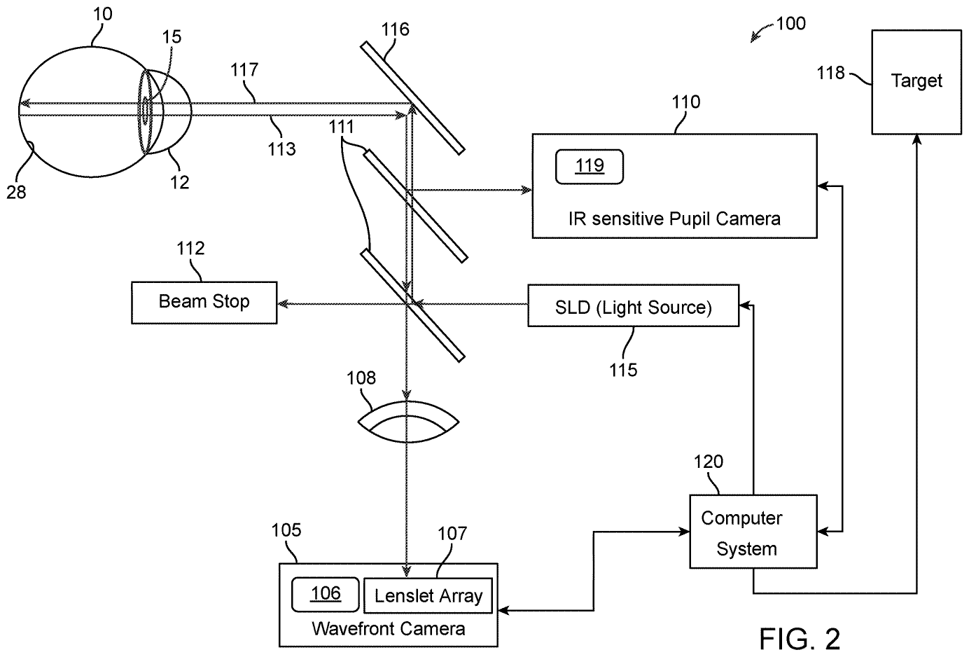

FIG. 2 illustrates in schematic an implementation of an optical system 100 that allows for objectively measuring refraction errors in an eye to assess accommodation of a lens in the eye. Generally, the system 100 includes a wavefront camera 105 and an infrared (IR) pupil camera 110. The wavefront camera 105 can continuously record a video of the Shack-Hartmann centroids during binocular, unimpeded viewing of a physical target 118 by the patient presented at known distances from the patient's eye 10. The pupil camera 110 and the wavefront camera 105 can be high-speed cameras operating continuously and synchronously with one another to record video of the patient's eye 10.

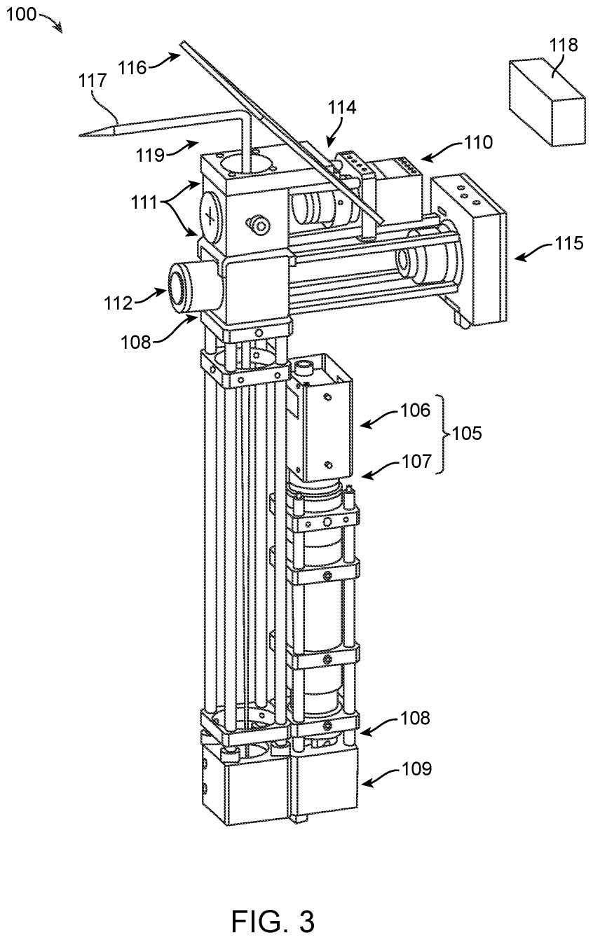

The pupil camera 110 of the optical system 100 can operate in two modes-forward-illumination mode and retro-illumination mode. In forward-illumination mode (i.e., conventional imaging), the pupil camera 110 uses an illuminating light source 119 of the system such as infrared LEDs. The illuminating light source 119 allows the operator to align the patient's pupil of the eye 10 being measured with the pupil camera 110 and the wavefront camera 105, which will be described in more detail below. In retro-illumination mode, the pupil camera 110 uses the same light source 115 as the wavefront camera 105, such as an infrared laser or a super-luminescent diode (SLD). In retro-illumination mode, light coming from the retina in a posterior-to-anterior direction (i.e., retro) is used to illuminate internal structures of the eye to resolve fine details in the internal optics for detection by the pupil camera 110 without the pupil camera 110 forming an image of the retina itself. As will be described in more detail below, the retro-illumination mode of the pupil camera 110 makes possible visualization and resolution of features in the eye's optics, such as the capsule, cataract, fibrosis, and many structural details of an implanted IOL. The pupil camera 110 can operate as an indexing camera that is capable of recording the eye throughout the entire measurement process so that output from the pupil camera 110 can be used to selectively analyze the data captured by the wavefront camera 105. The pupil camera 110 can be an IR-sensitive pupil camera including a 25 mm focal length pupil camera lens 114 (see FIG. 3). The wavefront camera 105 can be a Shack-Hartmann wavefront sensor comprising a sensor head 106 and microlens array 107. The wavefront camera 105 can be a high performance CCD+CMOS (GRASSHOPPER 3, Teledyne FLIR).

The two cameras 105, 110 have relatively high sampling rates that are synchronous with one another. The sampling rate for each can be at a frequency greater than about 10 Hz. In some implementations, the wavefront camera 105 and the pupil camera 110 can capture between 10 and 1000 frames per second, or between 10 and 100 frames per second, or between 20 and 50 frames per second. The cameras operate at the same time and the same rate allowing for spatial positioning in one camera (the pupil camera 110) to be translated onto the other camera (the wavefront camera 105) for narrowing the analytical range of the system 100. Narrowing the analytical range can allow for the full accommodative amplitude of an accommodating IOL to be captured.

“Synchronous” as used herein includes two or more cameras communicably coupled together (electrically and/or physically) having the same sampling rate where the frames are captured simultaneously in a time-synchronized manner. If the cameras were perfectly synchronous, the exposures would start and end exactly simultaneously. Cameras operating synchronously include cameras that are near synchronous or substantially synchronous so that the start and the end of exposure is within a tolerance of about 100 milliseconds difference from when the first pixel in the fame is captured. “Near synchronous” is understood to mean simultaneous operation within a tolerance of less than or equal to ±100 ms or less than or equal to ±10% of camera exposure duration.

The system 100 can include a measurement light source 115 such as an IR Laser or a super-luminescent diode (SLD) configured to provide the collimated light or probe beam 117 during a measurement. In some implementations, the measurement light source 115 has a center wavelength of 875 nm and a spectral bandwidth of 855-895 nm. The probe beam 117 from the measurement light source 115 is directed towards the eye 10 being measured. The beam 117 enters the eye 10 penetrating the cornea 12 and passing through the pupil 15 before reflecting off the retina 28. Reflected light or the wavefront 113 is directed back out the eye and towards the wavefront camera 105.

The SLD laser beacon can be very small in diameter (about 1 mm) so that it can more easily bypass anatomical features (e.g., pigment, posterior capsule opacification, edges of the lens capsule, cataract, etc.) or IOL structures (e.g., transitions between dynamic and static zones of the IOL, fluid channels within the IOL, etc.) that might blur or scatter the spot on the retina. The SLD laser beacon can be made intentionally very small in diameter so that it can be positioned to minimize and/or remove back-reflections from the optics of the crystalline lens or IOL in the patient. The beam position can be displaced relative to the optical axis of the system by adjusting an X-Y translation stage in the SLD collimating assembly to control how the beam 117 is directed into the eye. Another way to control how the beam 117 is directed into the eye is by moving the system relative to the patient's eye to direct the beam 117 through the desired part of the pupil (e.g., the location that generates the sharpest and brightest wavefront spots and minimizes back-reflections). The analysis software can be designed to measure the wavefront no matter where the image falls within the frame of the wavefront camera 105.

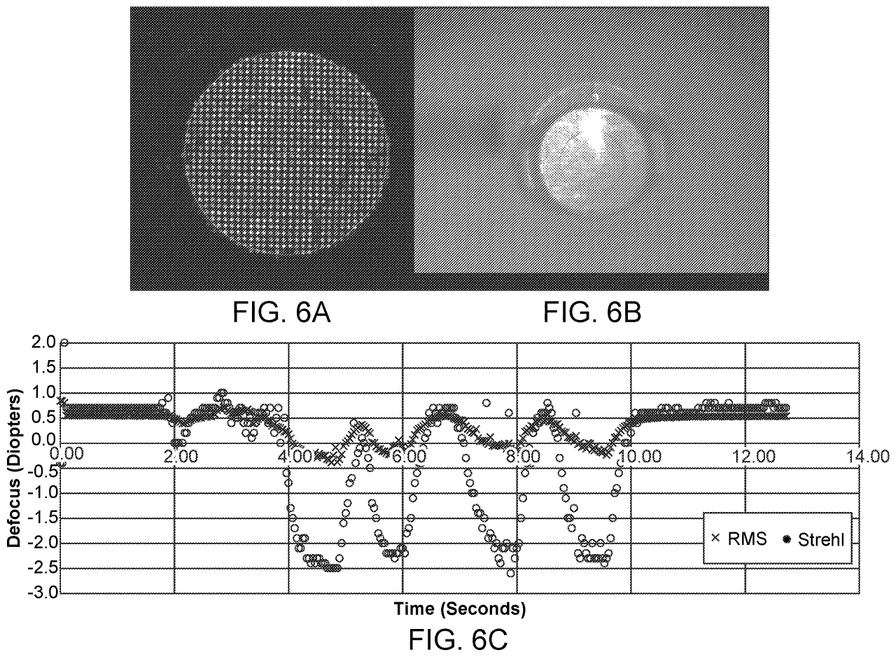

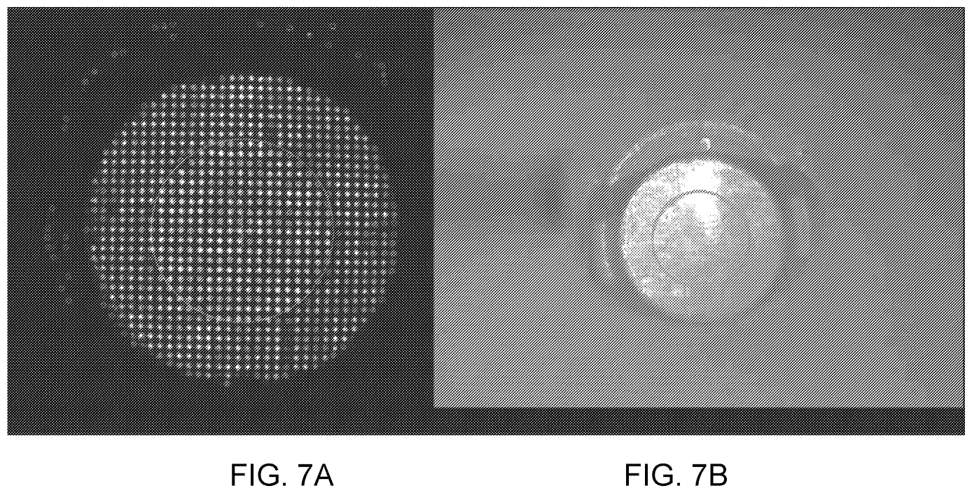

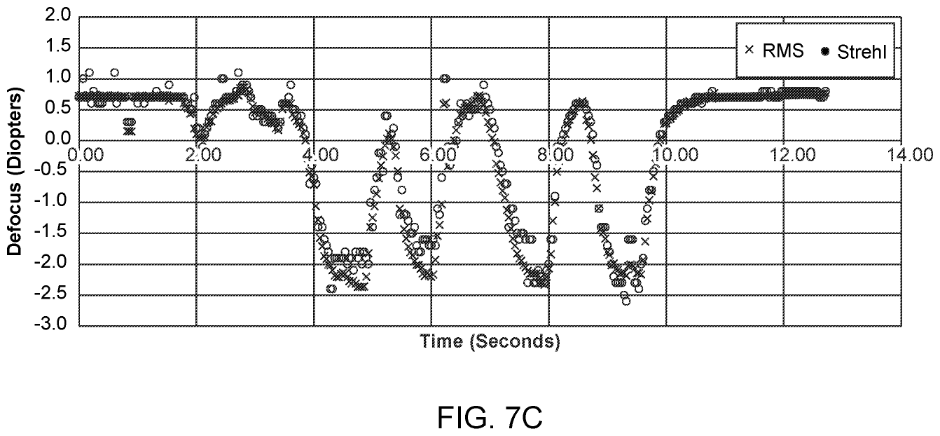

The optical system 100 can include one or more of lenses such as a collimating lens, polarizing beamsplitter, aperture, or other optical elements, etc. to direct light through the system 100. The measurement light source 115 can direct the probe beam 117 through a pellicle beam splitter 111 that reflects at least some light 117 towards the eye 10 and allows at least some light to pass through the beam splitter 111 to a beam stop 112. The pupil camera 110 can include a pellicle beam splitter 111 to reflect at least some of the wavefront 113 towards the pupil camera 110 and allow at least some light to pass through towards the wavefront camera 105. The wavefront 113 can pass through one or more achromatic doublet lenses 108 as it is directed towards the wavefront camera 105. In an implementation, the system 100 includes a first achromatic doublet lens 108 that is a 200 mm focal length achromatic doublet lens and a second achromatic doublet lens 108 that is a 150 mm focal length achromatic doublet lens. Light passing through the achromatic doublet lens 108 is directed through the microlens array 107 and captured by the sensor head 106 of the wavefront camera 105. The system 100 is shown in FIG. 3 as incorporating two 90 degree mirrors 109 to direct the wavefront 113 toward the wavefront camera 110, however, any of a variety of configurations is considered herein. The microlens array 107 can focus the distorted wavefront from the eye on the sensor head 106 of the wavefront camera 105. A spot field can be created on the camera 105. The intensity and location of each spot can then be analyzed to measure lower-and higher-order vision errors. FIGS. 6A and 7A show a frame from the wavefront camera illustrating the spot field.

FIG. 3 illustrates one configuration of the system 100 of FIG. 2. The system 100 can include a dichroic filter 116 such as a hot mirror positioned between the patient and the target 118 to reflect infrared light. The filter 116 is configured to reflect near infrared light and infrared light while transmitting visible light. Thus, the patient can view the target 118 with both eyes through the filter 116 while the filter 116 is used to reflect infrared light toward the cameras 105, 110. The filter 116 can be mounted near an upper end of the system 100 so that the patient can view the target 118 through the filter 116 over the top of the system 100. The target 118 can include a screen showing images, words, letters, and the like.

Although the patient views the target 118 with both eyes, the target 118 is aligned with a visual axis of the eye that is being measured. Aligning the target 118 with the eye 10 that is being measured avoids errors of measuring an eye in two separate converged states (i.e., straight, then converged). The targets 118 (distance and near) are aligned with one eye and the other eye is forced to converge. Measurements on the eye aligned with the targets 118 are recorded by the cameras 105, 110. This arrangement minimizes eye movement of the measured eye during the test and ensures the measurement is along the same optical axis, rather than one axis at distance and a second axis at near. Alignment of each target can include feedback from the patient although this is not necessary. For example, the operator rather than the patient can ensure the alignment prior to measurement. When properly positioned in the system, the patient is able to see light source 115. Because of the dichroic filter 116, the light source appears to be in visual field of the patient somewhere beyond the filter 116. Although the measurement light source 115 is visible to the patient as a small spot, the human eye is not very sensitive to near-infra-red light resulting in the spot being very dim and unlikely to distract the patient during actual measurement. The distance visual target can be positioned such that the target overlaps or nearly overlaps the apparent position of the light source 115 in the patient's visual field. Similarly, the near visual target can be positioned such that the target overlaps or nearly overlaps the apparent position of light source 115 in that patient's visual field. Such alignment will ensure that the near and distance targets are both positioned along the optical path of the system.

The system 100 can include an illuminating light source 119 such as an IR LED that allows the operator to align the patient's pupil of the eye 10 being measured with the pupil camera 110 and wavefront camera 105 as the patient views the target 118. In forward-illumination mode of the pupil camera 110, the illuminating light source 119 may be turned on during an initial spatial alignment or calibration steps of the system, but otherwise remains off during the measurement. In some implementations, the illuminating light source 119 can emit light at a wavelength that does not interfere with the measurement and thus, can remain on during a measurement. The illuminating light source 119 can project light onto the eye so that an operator can determine whether the patient's eye is aligned properly for the pupil and wavefront cameras. The light from the illuminating light source 119 as well as the light from the measurement light source 115 may be invisible to the operator, but can be seen on the pupil and wavefront cameras 105, 110. The illuminating light source 119 lights up the field so the operator can center the pupil in the frame and ensure appropriate focus for the test. When the pupil camera 110 is in forward-illumination mode, internal structures are generally not visible.

As discussed above, the system 100 allows for binocular open-viewing for unobtrusive measurement of the eyes. The system may incorporate a housing that rests on a base and having a touchscreen type display for the operator and one or more user interfaces, such as one for the patient and one for the operator. The patient interface may include one or more structures configured to hold a patient's head in a stable, immobile and preferably comfortable position during the measurements while also maintaining the eyes of the patient in a suitable alignment with the cameras. The patient may have their chin and/or forehead resting within or against one or more features of an alignment frame. The frame can be spatially adjusted to change the position of the patient's eye 10 to align the eye 10 with the cameras using the illuminating light source 119 as a guide. The illuminating light source 119 aligns along X and Y axes and also in Z focus. The eyes of the patient preferably remain in substantially the same position relative to the system 100 for all measurements performed. The frame can include a chin support and/or a forehead rest configured to hold the patient in a single, uniform position suitably aligned with respect to the system throughout the measurement. The patient may be seated in a chair that also can be adjusted to a suitable height relative to the patient interface.

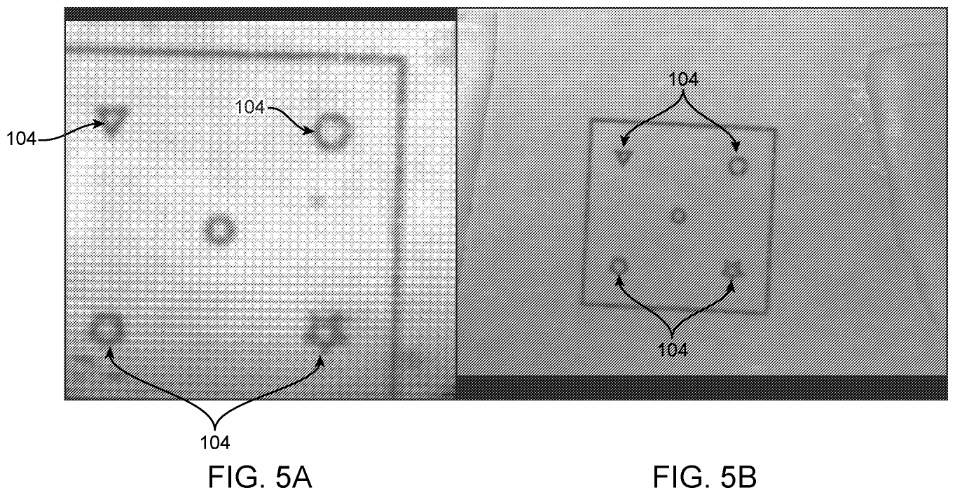

Calibration still frames can be captured by both cameras 105, 110 to calibrate the cameras 105, 110 to one another while the cameras are in the same alignment positions as they are while the videos are captured. The calibration still frames can be captured, preferably during the same session, prior to or after performing a test on a patient. The calibration frames remain valid so long as the camera positions and alignments are constant. The cameras 105, 110 can be calibrated to one another using any object that has two or more identifiable features when viewed through both cameras. In some implementations, the object can be a patient's eye such as a patient having an IOL implanted such that includes an identifiable object. The object can also be an item having text or marks such as a business card, ruler, paragraph of text, or printed card with specific graphics designed for this task. The object can be visible through both cameras so that the location of specific points on the object are identifiable. FIGS. 5A-5B illustrate one implementation of visible objects for calibrating the still frames. The calibration still frames are used to spatially map the frames captured by the wavefront camera 105 to the corresponding frames captured by the pupil camera 110, which will be described in more detail below.

A test on the first eye 10 can then be performed and the cameras 105, 110 operating to record raw data. A light beam 117 can be directed from the measurement light source 115 toward the first eye 10 to be measured and the wavefront 113 from the eye 10 captured by the wavefront camera 105. Infrared light from the eye 10 is captured by the pupil camera 110.

During the test, the patient views the target 118 binocularly through the filter 116. The target 118 can be set at a known distance in the patient's visual field while the cameras 105, 110 record the eye 10. The target 118 can then be moved to a second, closer distance while the cameras 105, 110 record the eye 10. Once the test is complete for the first eye 10, the alignment, calibration frame capture, and testing processes can be performed again for the other eye.

The videos capture raw data for each eye 10 from both cameras 105, 110 and can be analyzed using the software of the computer system 120 to calculate the patient's complete optical wavefront profile, which can be used to calculate refraction, accommodation, high order aberrations and pupil size. The patient's refraction can be correlated with the accommodative stimulus of the targets 118. Accommodative stimulus for a distance target 118 can be 0 for “distance” and the dioptric calculation based on the distance of the near target (1/distance in meters) such that 100 cm=1.0 D, 50 cm=2.0 D, 30 cm=3.33 D and so on. The refraction can be calculated according to known methods including the wavefront refraction method or the Strehl ratio method. The wavefront refraction method incorporates well-established formulas for describing a wavefront surface using Zernike polynomials and then converting the first few terms of that polynomial into a Sphere, Cylinder Refraction (see Thibos, L. N., Hong, X., Bradley, A., & Applegate, R. A. (2004). Accuracy and precision of objective refraction from wavefront aberrations. Journal of vision, 4(4), 9-9). The Spherical Equivalent (SE) can be calculated as SE=Sphere+0.5*Cyl. Rather than directly calculating optical power from the wavefront defocus RMS, the wavefront can be used to generate a series of point spread functions (PSF), which can be analyzed at varying levels of defocus. In determining the defocus level at which the Strehl Ratio of the PSF is maximized, a user can determine the “maximum Strehl Ratio” refraction for any given image. By plotting the “maximum Strehl Ratio” refraction across time and comparing change in refraction to the known target presentation sequence, the user can determine if the subject's refraction changes as a function of accommodative stimulus.

The patient can be presented with a pattern of targets at variable distances away from the patient in the examination room for intervals of time. The pattern, distances, and times can vary. The target can be moved between greater and smaller distances from the patient (e.g., 4.0 m to 0.4 m), each displayed for a select period of time (e.g., 3 seconds to 10 seconds), and the pattern repeating (e.g., 2, 3, 4, or more) over the course of a single test of an eye. The system may employ two or more discrete targets that are presented at various times during the test. The system may employ a single target whose position is adjusted during the test so as to provide a variable distance accommodative stimulus.

Infrared videography with a pupil camera conventionally measures pupil diameter and is used to locate the pupil center for indexing of the wavefront measurement and estimating the location of the visual axis. The pupil center and pupil diameter can change during a test, which can confound the analysis if the full pupil diameter is used for the analysis. Operators may err on the side of caution by selecting a zone for analysis that is slightly smaller than the full pupil diameter, which can omit important data points and/or negatively impact visual axis estimation.

Intraocular lenses (IOLs) implanted in one or both eyes of the patient may be detectable by the pupil camera 110 when in retro-illumination mode. Unlike the pupil, the IOL does not substantially change position or size during a measurement. Thus, the analysis of the raw data of the wavefront camera 105 can be indexed onto the IOL visualized by the pupil camera 110 rather than the pupil. The pupil is dynamic. The pupil changes in diameter, for example, by constricting in response to light and near fixation. The pupil center shifts slightly with pupil constriction. In contrast, the IOL is static and does not change in size during the test providing for a reliable indexing structure. Further, indexing the data to select regions of the IOL, such as the optical zone, can improve accuracy of the data analysis and allow for measurement of the full accommodative dynamics of the lens.

It should be appreciated, however, the system 100 can be indexed onto a natural structure such as the limbus or an iris feature, for example, if the patient has a natural lens and no IOL implanted. In this scenario, the system 100 may incorporate an additional measurement light source 119 (see FIG. 2) configured to illuminate the one or more natural structures of the eye for estimating the location of the visual axis. The additional measurement light source 119 can be at a wavelength that does not interfere with the wavefront measurement by the wavefront camera 105. Otherwise, the system 100 can incorporate a single measurement light source that is suitable for performing a measurement.

The pupil camera 110 when in retro-illumination mode can detect one or more specific structures of the IOL implanted within the eye 10 such as edges, visual marker(s), or other structural features of the IOL visible in an infrared pupil camera 110. The position of the IOL can be visualized in the video captured by the pupil camera 110 allowing for the operator to select a particular location of the IOL to focus the analysis of the video taken by the wavefront camera 105 and focus the analysis to the portion of the IOL that is of most interest (e.g., the accommodating diameter).

Some IOLs undergo shape change to provide accommodation for the patient, but only a selected diameter of the full optic diameter of the IOL actually changes shape. For example, if only the central 2.8 mm of the IOL changes shape, but the entire 4.0 mm diameter is used to analyze the change, the full extent of the power change is underestimated. The optical system 100 described herein is particularly useful for IOLs having a shape-changing dynamic portion that is smaller than the full optic diameter. The optical system 100 described herein is also used for IOLs whose shape change is proportionally concentrated in the center of the IOL. The optical system 100 described herein narrows the analytical range of the IOL to selected diameters to provide a more focused and accurate analysis of the video captured by the wavefront camera 105.

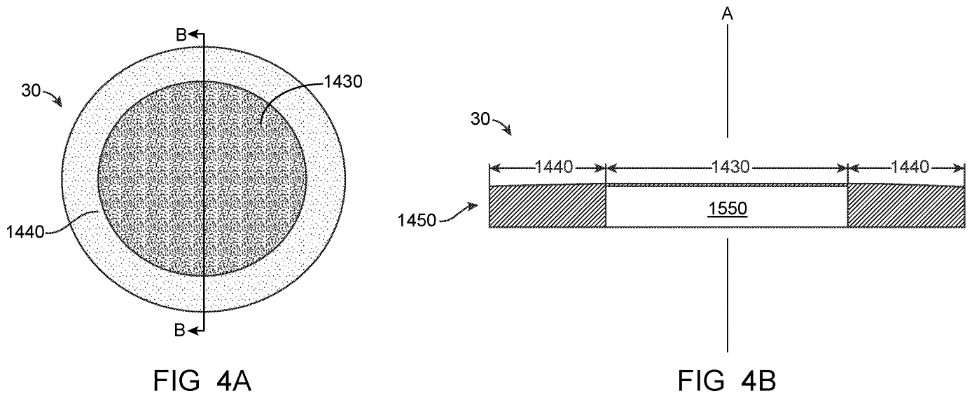

FIGS. 4A-4B illustrate in schematic partial views of an accommodating intraocular lens 30. The lens 30 can include an anterior optic 1450 having a central, dynamic zone formed by a dynamic membrane 1430 that is surrounded by a peripheral, static zone formed by a static anterior optical portion 1440. The dynamic membrane 1430 of the anterior optic 1450 is configured to undergo shape change for accommodation whereas the static anterior optical portion 1440 of the anterior optic 1450 is configured to resist shape change. The dynamic membrane 1430 can have a differential thickness gradient to provide precise control over the shape of the membrane 1430 and overall optical performance during shape change. The dynamic membrane 1430 can be designed to have different thickness gradients to provide a different membrane shape that provides the best optical performance for a particular AIOL. The thickness gradient across the dynamic membrane 1430 can be defined by the curvatures of the anterior (external) surface and the posterior (internal) surface of the dynamic membrane 1430, and in some implementations the curvature of the anterior surface of the static anterior optical portion 1440. The specific curvature combinations of the anterior surfaces and posterior surface of the dynamic membrane 1430 can provide improved optical quality.

The IOL 30 can incorporate a combination of solid and liquid optical components. The anterior optic 1450 can be a flexible optic formed of an optically clear, low modulus polymeric material such as silicone, polyurethane, or flexible acrylic. As mentioned above, the anterior optic 1450 can include a static anterior optical portion 1440 surrounding a central, dynamic membrane 1430 configured to outwardly bow as discussed elsewhere herein. The dynamic membrane 1430 can be positioned relative to the lens body such that the optical axis A of the lens extends through the dynamic membrane 1430. The anterior optic 1450 can have a variable thickness. For example, the dynamic membrane 1430 can have a reduced thickness compared to the static anterior optical portion 1440. The thinner cross-sectional thickness of the dynamic membrane 1430 compared to the cross-sectional thickness of the static anterior optical portion 1440 can render it relatively more prone to give way upon application of a force on its inner surface. For example, upon an increased force applied against inner surfaces of the anterior optic 1450 during deformation of the fluid chamber 1550, the dynamic membrane 1430 can bow outward along and coaxial to the optical axis A of the lens 30 while the static anterior optical portion 1440 maintains its shape. The dynamic membrane 1430 can be configured to give way due to pressure applied by the liquid optical material within the fluid chamber 1550 onto the internal surface of the anterior optic 1450 causing an outward bowing of the outer face (e.g., anterior face). Outer static anterior optical portion 1440 of the anterior optic 1450 can have a thickness greater than the inner dynamic membrane 1430 of the optic 1450 and can be more resistant to reshaping under such internal pressure applied by the liquid optical material in the fluid chamber 1550. The outer static anterior optical portion 1440 of the anterior optic 1450 can provide distance vision correction even when the inner dynamic membrane 1430 is reshaped for near vision. The dynamic membrane 1430 can also include multiple materials, for example, materials configured to flex near a center of the dynamic membrane 1430 and other materials configured to reinforce the optic zone and limit distortion. Thus, the dynamic membrane 1430 of the anterior optic 1450 can be formed of a material that is relatively more susceptible to outward bowing than the material of outer static anterior optical portion 1440. The various regions of the optic 1450 can be injection or compression molded to provide a relatively seamless and uninterrupted outer face. The material of the regions can be generally consistent, though the dynamic membrane 1430 can have different stiffness or elasticity that causes it to bow outward farther than the static anterior optical portion 1440.

The anterior optic 1450 can be configured to have varied multifocal capabilities to provide the wearer of the lenses described herein with enhanced vision over a wider range of distances, for example, as described in U.S. Publication No. 2009/0234449, which is incorporated by reference herein in its entirety. The “optic zone” as used herein generally refers to a region of the lens body that surrounds the optical axis A of the lens and is optically clear for vision. The “accommodating zone” as used herein generally refers to a region of the lens body capable of undergoing shape change for focusing (e.g. the dynamic membrane 1430). The optic zone is configured to have a corrective power although the entire optic zone may not have the same corrective power. For example, the dynamic membrane 1430 and the static anterior optical portion 1440 of the anterior optic may each be positioned within the optic zone. The dynamic membrane 1430 may have corrective power whereas the static anterior optical portion 1440 may not have corrective power. Or, for example, the diameter defined by the dynamic membrane 1430 may have an optical power and the static anterior optical portion 1440 may have a power that is greater or lesser than that of the dynamic membrane 1430. The dynamic membrane 1430 can be equal to or smaller than the overall optical zone can create a multi-focal lens. The accommodating zone of the lens body can be equal to or smaller than the overall optic zone.

Narrowing the analytical range of the wavefront camera 105 output to a particular region of interest in the eye, for example, just the accommodating dynamic membrane 1430 of the IOL 30, allows for an isolated analysis that captures the full accommodative amplitude of the IOL 30. In contrast, if only a small central diameter of an IOL changes shape, but the change is being averaged across the larger anterior diameter of the IOL, the full extent of the power change is not being captured in the analysis. The pupil camera 110 allows for the operator to select (or exclude) a particular location of the IOL 30 for analysis. The indexing is dependent on structures that do not change in diameter during accommodation and resolves the difficulties encountered by tracking the pupil or a part of the eye that changes in diameter during accommodation.

FIG. 5A illustrates one still frame captured by the wavefront camera 105 and FIG. 5B illustrates a corresponding still frame captured by the pupil camera 110. The still frames can be two images of the same object viewed through each camera 105, 110. Each frame is overlaid with spatial calibration markers 104 by software running on a computer system 120 of the system 100 so that a registration step may be performed prior to analysis of the data. The markers 104 can be positioned on identical locations of each corresponding frame so that the two outputs can be calibrated to one another even if the camera magnifications are different, as in FIGS. 5A-5B. Each of the markers 104 can be unique from one another, but identical between the corresponding frames. For example, FIG. 5A has 4 spatial markers 104 overlaid in different locations on the frame from the wavefront camera 105, each marker 104 having a different shape (triangle, circle, star, and pentagon). FIG. 5B has the same 4 spatial markers 104 overlaid on the corresponding frame from the pupil camera 110 in the same locations. A calibration protocol can be performed by an operator who selects each of the plurality of markers 104 projected on the frames such as by clicking each of the markers 104 with a mouse and using the software running on the computer system 120.

The recorded information from the pupil camera 110 can be used to analyze a select zone of the recorded information from the wavefront camera 105 to provide more accurate refractive data for the IOL. FIGS. 6A and 6B show frames from the wavefront camera 105 and the pupil camera 110, respectively. The spots of the lenslet array can get distorted or go missing near the location of the iris, which appears darker in each of the figures. FIGS. 7A and 7B show the same frames from the wavefront camera 105 and the pupil camera 110, respectively, as those in FIGS. 6A and 6B. The border between the central, dynamic optic portion 1430 of the IOL 30 and the static zone 1440 is identifiable in the pupil camera frame of FIG. 7B and can be used as a guide for selecting (or excluding or sub-selecting) the zone for analysis (see circle C drawn in FIG. 7B).

FIGS. 6C and 7C are plots of Defocus (D) vs. Time including Strehl defocus (o) and RMS defocus (x). FIG. 6C calculates defocus directly from the wavefront refraction and FIG. 7C is the defocus at which the Strehl ratio is maximized. In an emmetropic accommodating patient, the baseline level of defocus is 0.00 (D) when looking at a distance target. The patient's eye accommodates to focus on a near target presented at a selected distance, for example 3D for a target presented 33 cm away. The system measures that change in defocus. Patients can be presented with a known pattern (e.g., distance target for 5 seconds, near target for 5 seconds, distance target for 5 seconds, and near target for 5 seconds) and the measured defocus compared to determine whether the patient is accommodating as a response to the near target stimulus.

FIGS. 6C and 7C are plots from a bench test of an accommodating IOL as described herein. A force is manually applied to a perimeter region of the lens body to drive shape change of the anterior optic to simulate a near vision test. Releasing the force against the lens simulates a distance vision test. In the bench test, cycles of force applied to the lens can be performed similar to presenting known patterns to a patient during a test (e.g., no force for 3.5 s, force for 1 s, no force for 1 s, force for 1 s, no force for 1 s, then force for 2 s). Circle C is representative of the shape-changing dynamic portion of the IOL (e.g., dynamic membrane 1430) and excludes the static portion of the IOL near the perimeter of the anterior surface (e.g., optical portion 1440). FIG. 6C shows that when the full anterior surface of the IOL (shape changing region and non-shape changing region) was the selected zone for analysis, the RMS defocus had an attenuated signal of about 1.0 D. FIG. 7C shows that selecting circle C of FIG. 7B as the zone for analysis results in a full signal of about 3.0 D accommodation.

In some implementations, the pupil camera 110 frames are used to select the portion of the IOL that undergoes shape change so that the analysis of the wavefront data captures the full accommodative amplitude of the IOL. In other implementations, the pupil camera 110 frames are used to exclude a region of the IOL from the analysis of the wavefront data. In still further implementations, the pupil camera 110 frames are used to select a sub-region of the IOL that is configured to undergo shape change such that some shape change regions are included in the analysis and other shape change regions are excluded from the analysis.

Directing the beam(s), capturing the video, and acquiring the measurements are all under the direction of an overall computer system 120 of the system 100. The pupil camera 110 and the wavefront camera 105 can be coupled so that the location information included in the image and the measurement data can be associated for the analysis. The overall measurement of the aberrations throughout the optical system of the eye can be measured and the aberration data may be focused specifically to a particular source of the aberrations, via the pupil camera 110. The system 100 may execute an algorithm that removes data associated with reflections from the cornea and/or lens. Shape changing IOLs typically incorporate multiple optical interfaces. There is an increased risk of these reflections when performing a measurement on an IOL and thus, the reflections are preferably removed from the data so as to not contaminate the defocus calculation with false centroids. As mentioned above, the wavefront camera 105 can continuously record a video of the Shack-Hartmann centroids. The centroid of the Shack-Hartmann spot is estimated from the large amount of light that is scattered from the retinal layers recorded by the detector. False centroids look like Shack-Hartmann centroids, but are actually artifacts of reflection.

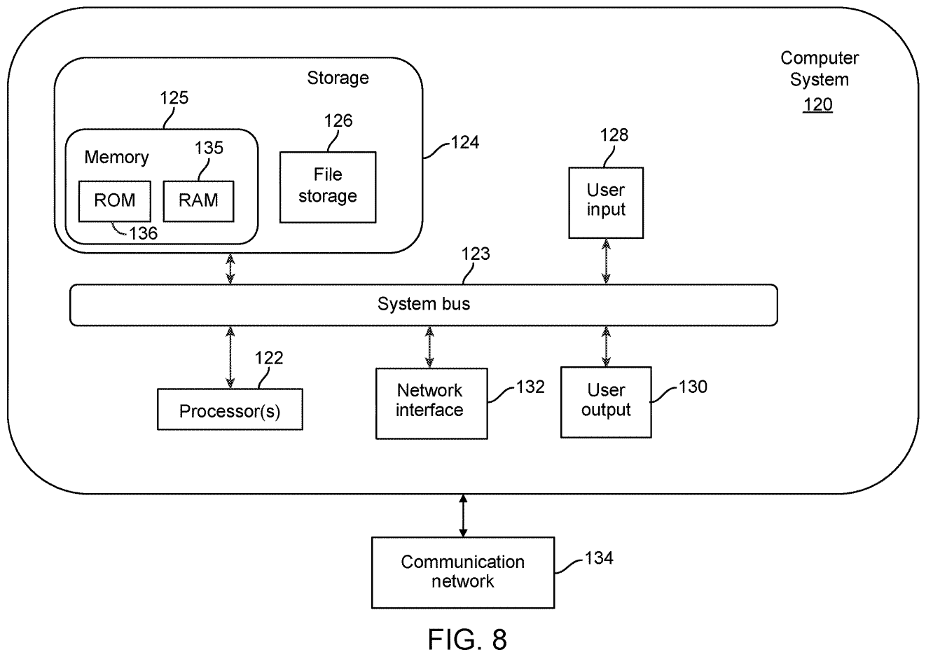

FIG. 8 is a simplified block diagram of an overall computer system 120 of the system 100. The computer system 120 can include at least one processor 122, which may communicate with one or more peripheral devices via a bus subsystem 123. The peripheral devices may include a storage subsystem 124 having a memory 125 and a file storage 126, user interface input device 128, user interface output device 130, and a network interface subsystem 132. The network interface subsystem 132 can provide an interface to outside networks 134 and/or other devices.

User interface input devices 128 may include a keyboard, pointing devices such as a mouse, trackball, touch pad, or graphics tablet, a scanner, foot pedals, a joystick, a touchscreen incorporated into the display, audio input devices such as voice recognition systems, microphones, and other types of input devices. User input devices 128 will often be used to download a computer executable code from a tangible storage media. In general, use of the term “input device” is intended to include a variety of conventional and proprietary devices and ways to input information into computer system 120.

User interface output devices 130 may include a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from computer system 120 to a user.

Storage subsystem 124 can store the basic programming and data constructs that provide the functionality of the system. For example, a database and modules implementing the functionality of the methods of the present invention, as described herein, may be stored in storage subsystem 124. These software modules are generally executed by processor 122. In a distributed environment, the software modules may be separated and stored on a plurality of computer systems and executed by processors of the plurality of computer systems. Storage subsystem 124 typically includes memory subsystem 125 and file storage subsystem 126.

Memory subsystem 125 typically includes a number of memories including a main random access memory (RAM) 135 for storage of instructions and data during program execution and a read only memory (ROM) 134 in which fixed instructions are stored. File storage subsystem 126 provides persistent (non-volatile) storage for program and data files, and may include tangible storage media, which may optionally embody wavefront sensor data, wavefront gradients, a wavefront elevation map, and the like. File storage subsystem 126 may include a hard disk drive, a floppy disk drive along with associated removable media, a Compact Digital Read Only Memory (CD-ROM) drive, an optical drive, DVD, CD-R, CD-RW, solid-state removable memory, and/or other removable media cartridges or disks. One or more of the drives may be located at remote locations on other connected computers at other sites coupled to computer system 120. The modules implementing the functionality of the system 100 may be stored by file storage subsystem 126.

Bus subsystem 123 provides a mechanism for letting the various components and subsystems of computer system 120 communicate with each other as intended. The various subsystems and components of computer system 120 need not be at the same physical location but may be distributed at various locations within a distributed network. Although bus subsystem 123 is shown schematically as a single bus, alternate embodiments of the bus subsystem may utilize multiple busses.

Computer system 120 itself can be of varying types including a personal computer, a portable computer, a workstation, a computer terminal, a network computer, a control system in a wavefront measurement system or laser surgical system, a mainframe, or any other data processing system. Due to the ever-changing nature of computers and networks, the description of computer system 120 depicted in FIG. 8 is intended only as an example for purposes of illustrating one embodiment of the present system. Many other configurations of computer system 120 are possible having more or less components than the computer system depicted in FIG. 8.

The wavefront camera 105 and the pupil camera 110 can each be in communication with and under the control of the computing system 120. The processor 122 of the computing system 120 can be in communication with a processor of the wavefront camera 105 and also a processor of the pupil camera 110. The wavefront data and pupil camera data may be stored in computer readable medium or memory of the computing system 120.

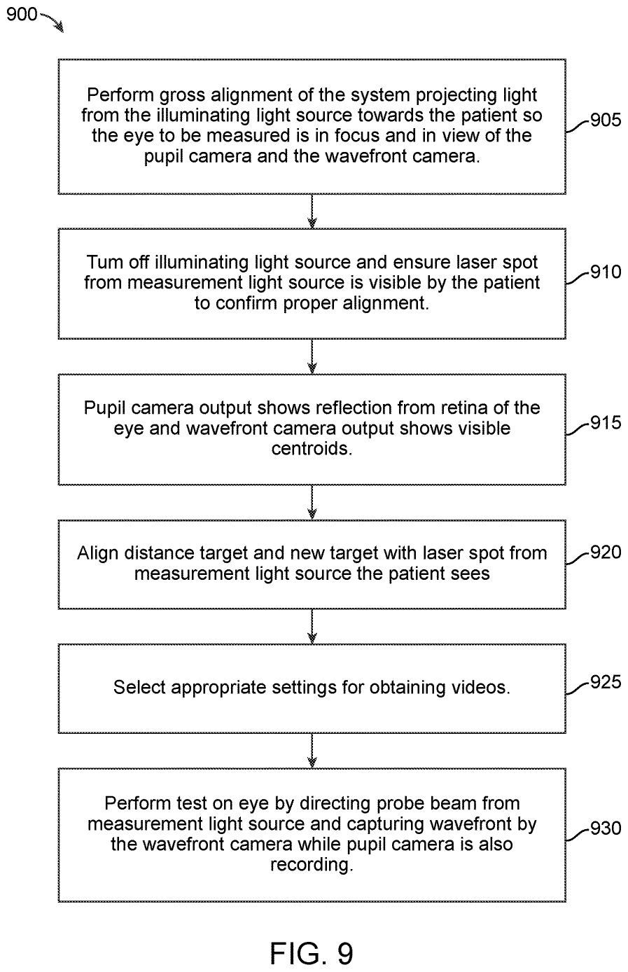

FIG. 9 illustrates a method 900 of using the system 100 described herein to perform a test on a patient. Gross alignment of the system is performed projecting light from the illuminating light source (e.g., LED) towards the patient so the eye to be measured is in focus and in view of the pupil camera and the wavefront camera (905). The illuminating light source is turned off and, if the system is properly aligned, the laser spot from the measurement light source is visible by the patient (910). The pupil camera output when in retro-illumination mode shows reflection of light from the retina of the eye and the wavefront camera output shows visible centroids (915). The distance target is aligned with the laser spot the patient sees and the near target is aligned with the laser spot the patient sees (920). The camera settings for each camera such as gain and exposure are selected as appropriate for obtaining videos (925). A test is performed on the first eye by directing a probe beam from the measurement light source toward the first eye and capturing the wavefront by the wavefront camera while the pupil camera is also recording (Step 930). Repeat each of the steps on the fellow eye to be measured. The pupil camera and the wavefront cameras are typically not adjusted during testing. Calibration still shots can be taken at any point and used to align the cameras to one another in case of unintentional misalignment of the system during use. The calibration still frame is a single frame from the video being captured by the pupil camera and a single frame from the video being captured by the wavefront camera that can be used for spatial calibration of the two cameras to each other.

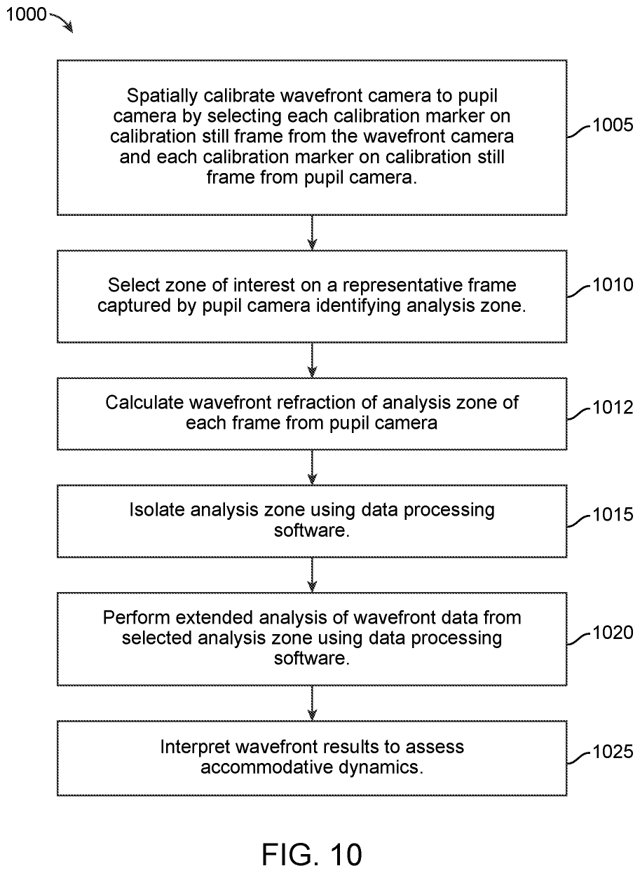

FIG. 10 illustrates a method 1000 of analyzing the data captured while performing the test of FIG. 9 to assess accommodative dynamics. The wavefront camera is spatially calibrated to the pupil camera by selecting each of the calibration markers on a calibration still frame from the wavefront camera and each of the calibration markers on a calibration still frame from the pupil camera (Step 1005). A zone of interest on a representative frame captured by the pupil camera is selected identifying an analysis zone (Step 1010). The wavefront refraction of the analysis zone of each frame of the video is calculated (Step 1012). Data processing software of the recorded videos isolates the analysis zone (Step 1015). The data processing software performs extended analysis of the wavefront data from the selected analysis zone (Step 1020). The wavefront results are interpreted to assess accommodative dynamics (Step 1025).

The optical system objectively measures accommodation in a patient and can include an infrared light source configured to provide retro-illumination light to a retina of an eye during a test; a wavefront camera configured to continuously record Shack-Hartmann centroids from the eye during the test providing a wavefront camera output; and an infrared-sensitive pupil camera configured to continuously record video of the eye from the retro-illumination light returned from the eye during the test providing a pupil camera output. The wavefront camera and the pupil camera are operatively coupled to synchronously or near synchronously operate at a sampling rate during the test.

The wavefront camera and the pupil camera can operate synchronously within a tolerance of less than 100 milliseconds. The optical system can include a computer system operatively coupling the pupil camera and the wavefront camera. The computer system can be programmed to index the wavefront camera output to an analytical zone of the pupil camera output independent of a pupil diameter of the eye. The computer system can be programmed to index the wavefront camera output to a structure of the eye visible on the pupil camera output. The structure of the eye preferably does not change size during the test. The structure of the eye can include a man-made structure implanted in the eye including at least a portion of an intraocular lens or a natural structure of the eye including a limbus. The sampling rate can be at a frequency greater than about 10 Hz. The infrared light source can be an infrared laser or a super-luminescent diode. The infrared light source can be configured to provide a collimated light or probe beam during the test. The infrared light source can include a center wavelength of about 875 nm and a spectral bandwidth of about 855 nm-895 nm. The optical system can include a physical target positionable a distance away from the eye and visible by the patient under binocular, unimpeded viewing. The optical system can further include a dichroic filter positioned between the patient and the physical target. The dichroic filter can be a hot mirror.

A method of using an optical system to assess patient refraction including a step of directing infrared light from an infrared light source onto a retina of an eye during a test; continuously recording Shack-Hartmann centroids from the eye with a wavefront camera at a sampling rate during the test providing a wavefront camera output; and continuously recording video of the eye from the retro-illumination light returned from the eye at the sampling rate during the test by an infrared-sensitive pupil camera providing a pupil camera output.

The method can further include the step of obtaining a calibration still shot of the eye from the pupil camera and obtaining a calibration still shot of the eye from the wavefront camera. The method can further include the step of selecting an optical zone of interest from the pupil camera output using data processing software; and interpreting wavefront results to assess accommodative dynamics based on the optical zone of interest. The patient can be pseudophakic and have an intraocular lens. The intraocular lens can be monofocal, multifocal, EDOF, Toric, or accommodating. The patient can be phakic.

In an interrelated method of assessing patient refraction, the method includes the step of performing gross alignment of an optical system having a physical target positionable during a test between a first distance and a second distance; an illuminating light source; a measurement light source configured to project a laser spot; an infrared-sensitive pupil camera; and a wavefront camera. The method includes steps of projecting light from the illuminating light source of the optical system towards a patient so an eye to be measured is in view of the pupil camera and the wavefront camera; aligning the distance target with the laser spot visible to the patient; aligning the near target with the laser spot visible to the patient; performing the test on the eye while operating the wavefront camera and the pupil camera at a sampling rate to obtain pupil camera output and wavefront camera output that is near synchronous.

The step of performing the test can include positioning the physical target for a period of time at the first distance and positioning the physical target for a period of time at the second distance while obtaining the pupil camera output and the wavefront camera output. The method can further include a step of spatially calibrating the pupil camera output to the wavefront camera output to narrow an analytical range of the system to capture a full accommodative amplitude of the eye. The step of performing the test can include directing a probe beam from the measurement light source and capturing a wavefront by the wavefront camera while the pupil camera records the eye. The method can further include a step of performing a test on a fellow eye. The method can further include a step of selecting calibration markers on a calibration still frame captured from the wavefront camera output and selecting calibration markers on a calibration still frame captured from the pupil camera output. The method can further include a step of selecting a zone of interest on a representative frame captured from the pupil camera output to identify an analysis zone. The method can further include a step of isolating the analysis zone using data processing software. The method can further include a step of performing extended analysis of wavefront data from the analysis zone selected using the data processing software. The method can further include a step of interpreting wavefront results to assess accommodative dynamics of the eye. The step of performing gross alignment can include aligning the physical target with a visual axis of the eye being measured. The physical target can be aligned with the eye being measured and the fellow eye converges.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “an aspect,” “one aspect,” “one implementation, “an implementation,” or the like, means that a particular feature, structure, configuration, or characteristic described is included in at least one embodiment, aspect, or implementation. Thus, the appearance of the phrase “one embodiment,” “an embodiment,” “one aspect,” “an aspect,” “one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. An optical system to objectively measure accommodation in a patient, the system comprising:
an infrared light source configured to provide retro-illumination light to a retina of an eye during a test;
a wavefront camera configured to continuously record Shack-Hartmann centroids from the eye during the test providing a wavefront camera output; and
an infrared-sensitive pupil camera configured to continuously record video of the eye from the retro-illumination light returned from the eye during the test providing a pupil camera output,
wherein the wavefront camera and the pupil camera are operatively coupled to synchronously or near synchronously operate at a sampling rate during the test.

2. The optical system of claim 1, wherein the wavefront camera and the pupil camera operate synchronously within a tolerance of less than 100 milliseconds.

3. The optical system of claim 1, further comprising a computer system operatively coupling the pupil camera and the wavefront camera, wherein the computer system is programmed to index the wavefront camera output to an analytical zone of the pupil camera output independent of a pupil diameter of the eye.

4. The optical system of claim 2, wherein the computer system is programmed to index the wavefront camera output to a structure of the eye visible on the pupil camera output.

5. The optical system of claim 4, wherein the structure of the eye does not change size during the test.

6. The optical system of claim 4, wherein the structure of the eye comprises a man-made structure implanted in the eye including at least a portion of an intraocular lens or a natural structure of the eye including a limbus.

7. The optical system of claim 1, wherein the sampling rate is at a frequency greater than about 10 Hz.

8. The optical system of claim 1, wherein the infrared light source is an infrared laser or a super-luminescent diode.

9. The optical system of claim 1, wherein the infrared light source is configured to provide a collimated light or probe beam during the test.

10. The optical system of claim 1, wherein the infrared light source comprises a center wavelength of about 875 nm and a spectral bandwidth of about 855 nm-895 nm.

11. The optical system of claim 1, further comprising a physical target positionable a distance away from the eye and visible by the patient under binocular, unimpeded viewing.

12. The optical system of claim 11, further comprising a dichroic filter positioned between the patient and the physical target.

13. The optical system of claim 12, wherein the dichroic filter is a hot mirror.

14. A method of using an optical system to assess patient refraction comprising:
directing infrared light from an infrared light source onto a retina of an eye during a test;
continuously recording Shack-Hartmann centroids from the eye with a wavefront camera at a sampling rate during the test providing a wavefront camera output; and
continuously recording video of the eye from the retro-illumination light returned from the eye at the sampling rate during the test by an infrared-sensitive pupil camera providing a pupil camera output.

15. The method of claim 14, further comprising obtaining a calibration still shot of the eye from the pupil camera and obtaining a calibration still shot of the eye from the wavefront camera.

16. The method of claim 15, further comprising selecting an optical zone of interest from the pupil camera output using data processing software; and interpreting wavefront results to assess accommodative dynamics based on the optical zone of interest.

17. The method of claim 14, wherein the patient is pseudophakic and has an intraocular lens.

18. The method of claim 17, wherein the intraocular lens is monofocal, multifocal, EDOF, Toric, or accommodating.

19. The method of claim 14, wherein the patient is phakic.

20. A method of assessing patient refraction comprising:
performing gross alignment of an optical system comprising:
a physical target positionable during a test between a first distance and a second distance;
an illuminating light source;
a measurement light source configured to project a laser spot;
an infrared-sensitive pupil camera; and
a wavefront camera;
projecting light from the illuminating light source of the optical system towards a patient so an eye to be measured is in view of the pupil camera and the wavefront camera;
aligning the distance target with the laser spot visible to the patient;
aligning the near target with the laser spot visible to the patient; and
performing the test on the eye while operating the wavefront camera and the pupil camera at a sampling rate to obtain pupil camera output and wavefront camera output that is near synchronous.

21.-31. (canceled)
